(19) United States
(12) Patent Application Publication
(10) Pub. No.: US 2013/0261626 A1
(43) Pub. Date: Oct. 3, 2013

(54) ORTHOPAEDIC SURGICAL INSTRUMENT FOR REMOVING AN IMPLANTED HUMERAL STEM COMPONENT AND METHOD OF USING THE SAME

(71) Applicants: Jason M. Chavarria, Warsaw, IN (US); Kyle E. Lappin, Fort Wayne, IN (US); Patrick G. McElhaney, JR., Juniper, FL (US)

(72) Inventors: Jason M. Chavarria, Warsaw, IN (US); Kyle E. Lappin, Fort Wayne, IN (US); Patrick G. McElhaney, JR., Juniper, FL (US)

(21) Appl. No.: 13/434,304

(22) Filed: Mar. 29, 2012

(51) Int. Cl. A61B 17/16

(52) U.S. Cl. 606/79

(57) ABSTRACT

A surgical instrument for removing an implanted humeral stem component from the humerus of a patient includes an offset surgical tamp. A surgical method for the use of such a surgical instrument is also disclosed.

### TECHNICAL FIELD

The present disclosure relates generally to an orthopaedic instrument for use in the performance of an orthopaedic joint replacement procedure, and more particularly to an orthopaedic surgical instrument assembly for removing an implanted humeral stem component.

### BACKGROUND

During the lifetime of a patient, it may be necessary to perform a total shoulder replacement procedure on the patient as a result of, for example, disease or trauma. In a total shoulder replacement procedure, a humeral prosthesis having a prosthetic head is used to replace the natural head of the patient's humerus. The humeral prosthesis typically includes an elongated humeral stem component that is implanted into the intramedullary canal of the patient's humerus. In such a total shoulder replacement procedure, the natural glenoid surface of the scapula is resurfaced or otherwise replaced with a glenoid component that provides a bearing surface upon which the prosthetic head of the humeral prosthesis articulates.

From time-to-time, revision surgery is performed to replace a previously-implanted humeral stem component. In such a revision surgery, the previously implanted humeral stem component is surgically removed and a replacement humeral stem component is implanted in the patient's humerus.

### SUMMARY

According to one aspect, a method of surgically removing an implanted humeral stem component from the humerus of a patient includes cutting a bone fragment from a medial surface of the humerus of the patient so as to expose an inferior surface of a collar of the humeral stem component. An engagement tip of a surgical tamp in then positioned contact with the inferior surface of the collar of the humeral stem component. The surgical tamp is then impacted so as to urge the humeral stem component from the humerus of the patient.

An osteotomy may be performed on the medial surface of the humerus of the patient to cut the bone fragment from the medial surface of the humerus of the patient. Such an osteotomy may be performed by advancing an osteotome into the medial surface of the humerus of the patient at a location under an inferior edge of the collar of the humeral stem component.

The surgical tamp may include a handle that is offset from the engagement tip. In such an embodiment, the handle of the surgical tamp is impacted along a first axis so as to apply an extraction force to the humeral stem component along a second axis. The first axis being parallel to, and spaced apart from, the second axis.

The engagement tip of the surgical tamp has a slot formed therein such that a medial fin of the humeral stem component is received into the slot.

According to another aspect, a method of surgically removing an implanted humeral stem component from a humerus of a patient includes positioning an engagement tip of an offset surgical tamp in contact with an inferior surface of the collar of the humeral stem component. The surgical tamp may be impacted along a first axis so as to apply an extraction force to the humeral stem component along a second axis. The first axis is parallel to, and spaced apart from, the second axis.

In some embodiments, the method may also include cutting a bone fragment from a medial surface of the humerus of the patient so as to expose the inferior surface of the collar of the humeral stem component. An osteotomy may be performed on the medial surface of the humerus of the patient to cut the bone fragment from the medial surface of the humerus of the patient. Such an osteotomy may be performed by advancing an osteotome into the medial surface of the humerus of the patient at a location under an inferior edge of the collar of the humeral stem component.

The surgical tamp may include a handle that is offset from the engagement tip. In such an embodiment, the handle of the surgical tamp is impacted along the first axis so as to apply the extraction force to the humeral stem component along the second axis. The engagement tip of the surgical tamp may have a slot formed therein such that a medial fin of the humeral stem component is received into the slot.

In some embodiments, positioning the engagement tip of the surgical tamp in contact with the inferior surface of the collar of the humeral stem component may include impacting the surgical tamp along a third axis extending orthogonal to a longitudinal axis of the humeral stem component.

According to another aspect, an orthopaedic surgical instrument for extracting an implanted humeral stem component for a patient's humerus includes a surgical tamp. The surgical tamp includes an elongated shaft having a proximal end and an opposite, distal end. A strike plate is secured to the proximal end of the elongated shaft, with an engagement tip being secured to the distal end of the elongated shaft. The proximal end defines a first longitudinal axis, with the distal end defining a second longitudinal axis. The second axis is offset from and parallel to the first axis.

The elongated shaft of the surgical tamp may further include a mid-shaft section connected to and extending distally from the proximal end, a first elbow connected to the mid-shaft section, and a second elbow connected to both the first elbow and the distal end of the elongated shaft.

The surgical tamp may also include a grip positioned around the elongated shaft at a location proximate to the strike plate.

The engagement tip may define a third longitudinal axis which is orthogonal to both the first and second longitudinal axes.

The second axis is offset from the first axis in a direction that is orthogonal to both the first and second axes.

The engagement tip of the surgical tamp may have a slot formed therein.

### BRIEF DESCRIPTION OF THE DRAWINGS

The detailed description particularly refers to the following figures, in which:

FIG. 1 is a perspective view of an orthopaedic surgical tamp that may be used in a surgical procedure to extract an implanted humeral stem component;

FIG. 2 is a cross-sectional view of the surgical tamp taken along the line 2-2 of FIG. 1, as viewed in the direction of the arrows;

FIG. 3 is a perspective view showing a humeral stem component implanted in the humerus of a patient;

FIG. 4 is a view similar to FIG. 3, but showing a bone fragment having been removed from a medial side of the patient's humerus so as to expose an inferior surface of the collar of the humeral stem component;

FIG. 5 is a fragmentary elevational view showing the surgical tamp engaged with the inferior surface of the collar of the humeral stem component;

FIG. 6 is an enlarged fragmentary perspective view showing the surgical tamp engaged with the inferior surface of the collar of the humeral stem component;

FIG. 7 is an enlarged fragmentary elevational view showing the surgical tamp engaged with the inferior surface of the collar of the humeral stem component, note that a portion of the humerus has been cutaway in the drawing of FIG. 7 for clarity of description;

FIG. 8 is a view similar to FIG. 6, but showing the humeral stem component being removed from the humerus of the patient;

FIG. 9 is a view similar to FIG. 7, but showing the humeral stem component being removed from the humerus of the patient;

FIG. 10 is an enlarged fragmentary perspective view showing another embodiment of an orthopaedic surgical tamp and the collar of the humeral stem component;

FIG. 11 is a fragmentary elevational view showing the surgical tamp of FIG. 10 impacted into the humerus of the patient;

FIG. 12 is a view similar to FIG. 10 showing the surgical tamp engaged with the inferior surface of the collar of the humeral stem component; and

FIG. 13 is a fragmentary elevational view showing the surgical tamp engaged with the inferior surface of the collar of the humeral stem component.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention.

Terms representing anatomical references, such as anterior, posterior, medial, lateral, superior, inferior, etcetera, may be used throughout this disclosure in reference to both the orthopaedic implants described herein and a patient's natural anatomy. Such terms have well-understood meanings in both the study of anatomy and the field of orthopaedics. Use of such anatomical reference terms in the specification and claims is intended to be consistent with their well-understood meanings unless noted otherwise.

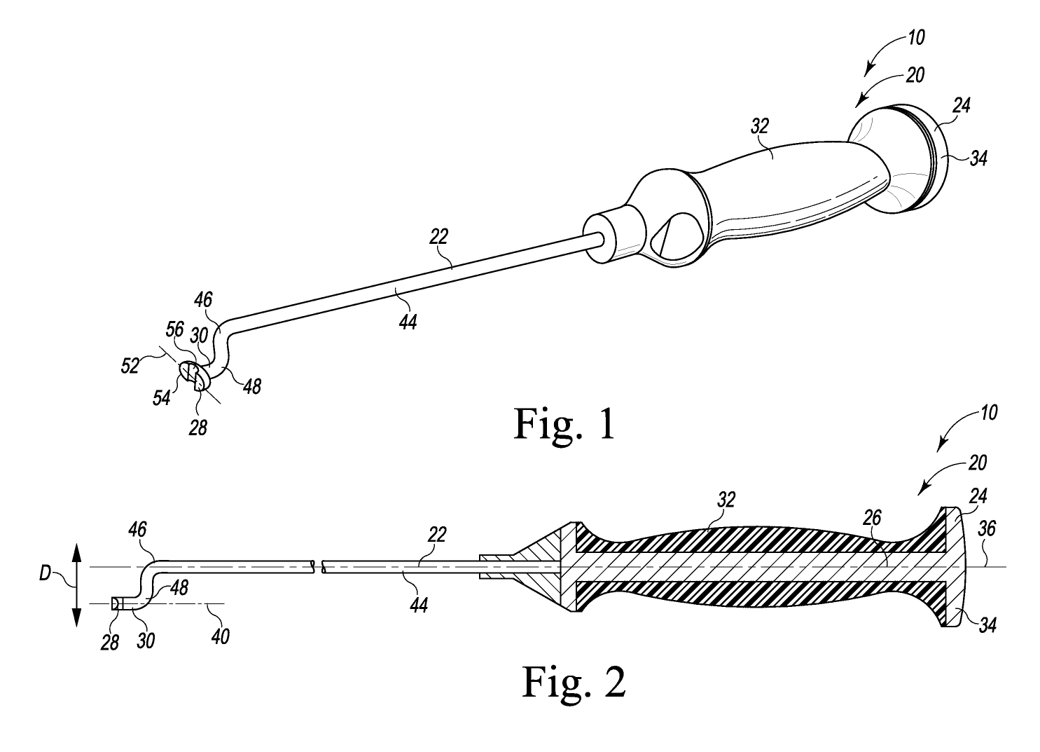

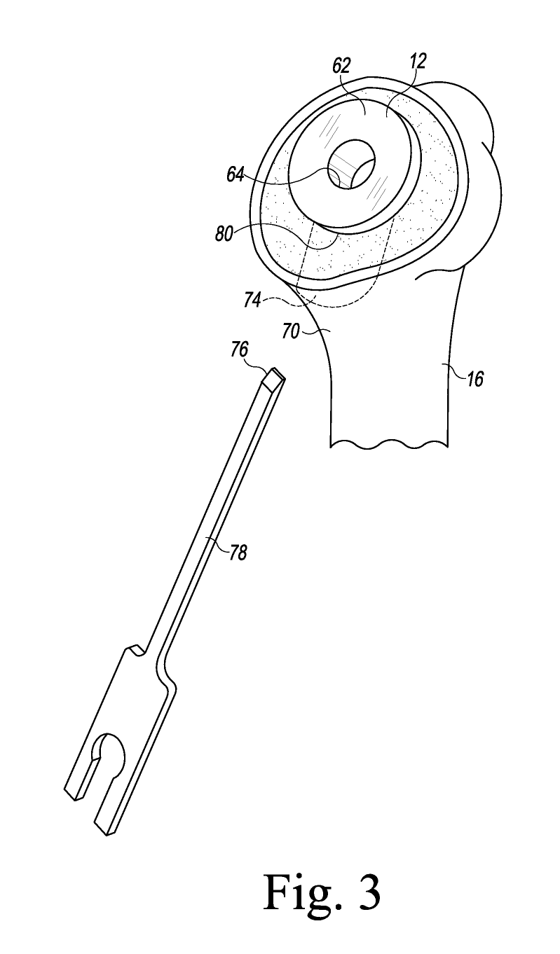

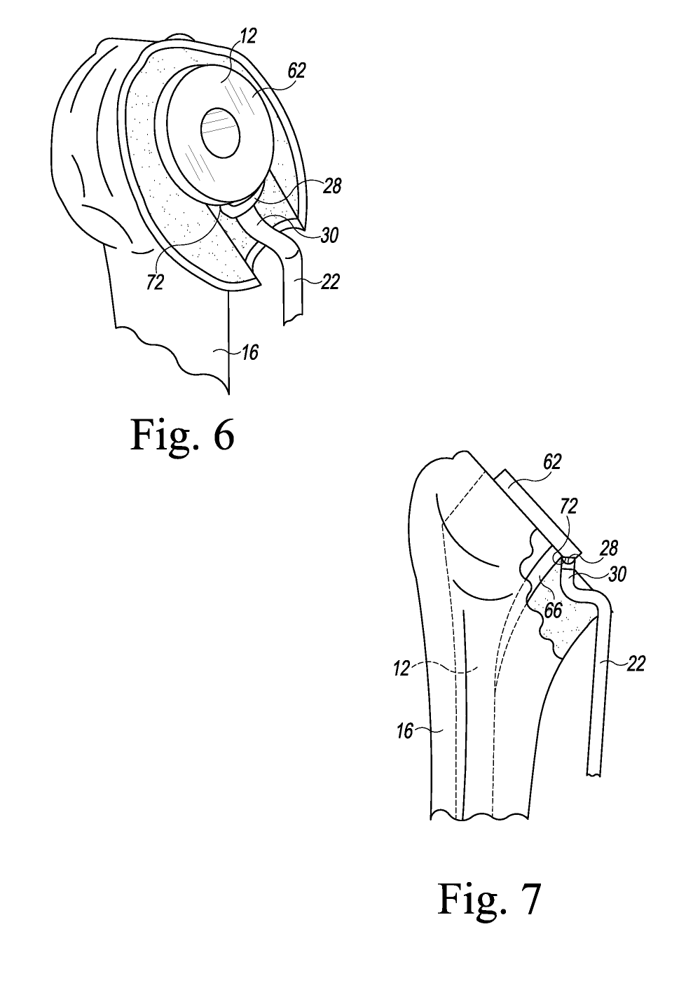

Referring now to FIGS. 1 and 2, there is shown an orthopaedic surgical instrument 10 for removing an implanted humeral stem component 12 from the intramedullary canal 14 of the humerus 16 of a patient (see also FIGS. 3-9). The surgical instrument 10 is embodied as an offset surgical tamp 20. The offset surgical tamp 20 includes an elongated shaft 22 having an impact head 24 on its proximal end 26 and an engagement tip 28 on its distal end 30. A sleeve or grip 32 is positioned around, and immovably coupled to, the outer surface of the surgical tamp's shaft 22 such as by, for example, overmolding. The sleeve 32 functions as a grip for allowing the surgeon to hold the offset surgical tamp 20 during a surgical procedure to extract the humeral stem component 12 from the patient's humerus 16.

The impact head 24 of the offset surgical tamp 20 includes a circular metal strike plate 34. In use, the surgeon holds the offset surgical tamp 20 via the grip 32 and strikes the metal strike plate 34 with a surgical mallet, sledge, or other impaction tool to generate an extraction force to extract the humeral stem component 12 from the patient's humerus 16.

As can be seen best in FIG. 2, the proximal end 26 of the surgical tamp's elongated shaft 22 is offset from its distal end 30. In particular, the proximal end 26 of the elongated shaft 22 has a longitudinal axis 36, with the distal end 30 of the shaft having a different longitudinal axis 40 that is offset from, and parallel to, the longitudinal axis 36 of the surgical tamp's proximal end 26. In the illustrative embodiment described herein, the longitudinal axis 40 of the distal end 30 of the shaft 22 is offset from the longitudinal axis 36 of the surgical tamp's proximal end 26 in a direction D that is orthogonal to both axes 36, 40.

Such an offset geometry is accomplished by a pair of 90° elbows formed in the elongated shaft 22. In particular, the proximal end 26 of the shaft 22 transitions to a mid-shaft section 44 that extends distally away from the proximal end 26 and, in turn, transitions to an elbow 46. The elbow 46 extends orthogonally away from the mid-shaft section 44 and transitions to another elbow 48. The elbow 48, in turn transitions to the distal end 30 of the elongated shaft 22.

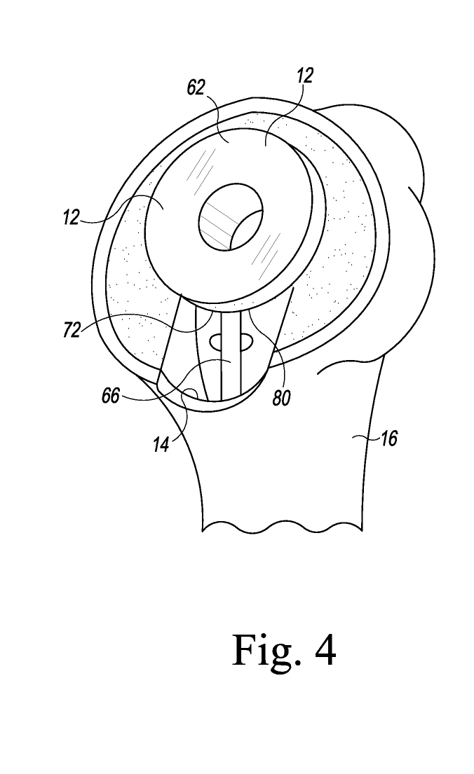

As can be seen in FIG. 1, the engagement tip 28 is arcuate in shape and extends in a direction that is orthogonal to the distal end 30 of the elongated shaft 22. In particular, the engagement tip has a longitudinal axis 52 that is orthogonal to the longitudinal axis 40 of the distal end 30 of the shaft 22. The engagement tip 28 has a generally planar engaging face 54 having a slot 56 formed therein. The slot 56 allows the engagement tip 28 to straddle a medial fin 66 of the humeral stem component 12 (see FIG. 4).

The metallic components of offset surgical tamp 20 (e.g., the elongated shaft 22 and the strike plate 34) may be constructed from a medical-grade metal such as stainless steel, cobalt chrome, or titanium, although other metals or alloys may be used. Moreover, in some embodiments, rigid polymers such as polyaryetheretherketone (PEEK) may also be used. The grip 32 may be constructed from a polymer such as silicone.

Referring now to FIGS. 3-9, there is shown a revision surgical procedure in which the surgical instrument 10 is used to surgically extract (i.e., remove) a previously implanted humeral stem component 12 from the intramedullary canal 14 of the humerus 16 of a patient. As can be seen in FIG. 3, in a previous primary procedure, the humeral stem component 12 was implanted in the patient's humerus 16. A typical humeral stem component 12 includes an annular-shaped collar 62 having a tapered bore 64 formed therein to receive a tapered stem of spherical head component (not shown). The collar 62 is positioned on a substantially planar surgically-resected humeral surface formed during the primary procedure. The collar 62 is formed on the superior surface of the body of the humeral stem component 12. A medial fin 66 extends along the medial surface of the humeral stem component 12, as shown in FIG. 4.

During a revision surgery, it may be necessary to remove the previously implanted humeral stem component 12 from the patient's humerus 16. As shown in FIGS. 3-6, the offset surgical tamp 20 may used to surgically remove a previously implanted humeral stem component 12.

To do so, a bone fragment is first cut from a medial surface 70 of the humerus 16 of the patient so as to expose an inferior surface 72 of the collar 62 of the implanted humeral stem component 12. As shown in FIG. 3, one way to cut the bone in such a manner is to perform an osteotomy on the medial surface 70 of the humerus 16 of the patient so as to cut the bone fragment 74 (shown in phantom in FIG. 3 prior to removal) from the medial surface 70 of the humerus 16. To do so, the cutting tip 76 of an osteotome 78 is advanced into the medial surface 70 of the humerus 16 of the patient at a location under an inferior edge 80 of the collar 62 of the humeral stem component 12. The motion of the cutting tip 76 causes the bone fragment 74 to be cut and thereafter removed. The bone fragment 74 may be saved for re-implantation later in the surgical procedure.

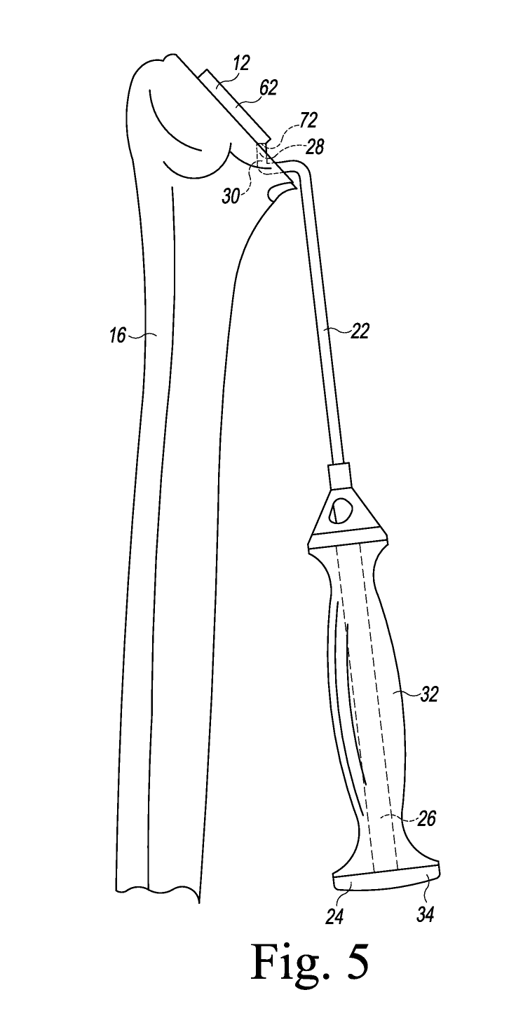

Once the inferior surface 72 of the collar 62 of the implanted humeral stem component 12 has been exposed, the surgeon may then use the offset surgical tamp 20 to extract the humeral stem component 12. To do so, as shown in FIGS. 5-7, the surgeon first advances the surgical tamp 20 such that its engagement tip 28 is positioned in contact with the inferior surface 72 of the collar 62 of the implanted humeral stem component 12. During such positioning of the engagement tip 28, the medial fin 66 of the humeral stem component 12 is received into the slot 56 thereby allowing the engagement tip 28 to straddle a medial fin 66 and firmly engage the inferior surface 72 of the collar 62.

Once the engagement tip 28 is positioned against the inferior surface 72 of the collar 62 in such a manner, the surgeon strikes the metal strike plate 34 with a surgical mallet, sledge, or other impaction tool to generate an extraction force that is applied to the inferior surface 72 of the collar 62. As can be seen in FIG. 5, because of the offset design of the offset surgical tamp 20 the extraction force is transmitted to the collar 62 along a different axis than as applied to the strike plate 34. In particular, the surgeon impacts the metal strike plate 34 along an axis that corresponds to the longitudinal axis 36 of the proximal end 26 of the elongated shaft 22. However, the resulting extraction force is applied to the inferior surface 72 of the collar 62 along an axis that corresponds to the longitudinal axis 40 of the distal end 30 of the shaft 22.

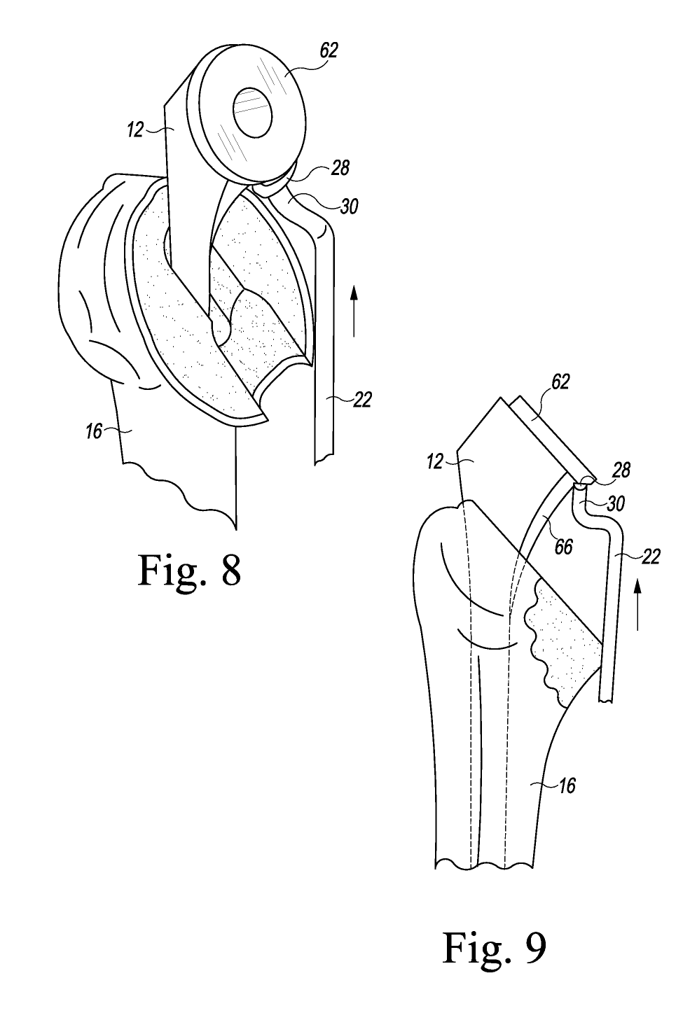

Repeated strikes from the surgeon onto the metal strike plate 34 of the handle urges the implanted humeral stem component 12 from the patient's humerus 16 as shown in FIGS. 8 and 9. The surgeon may then lift away the freed humeral stem component 12 from the patient's humerus 16. The surgeon may then perform the remainder of the revision surgical procedure, including the implantation of a revision humeral stem component (not shown) into the patient's humerus 16.

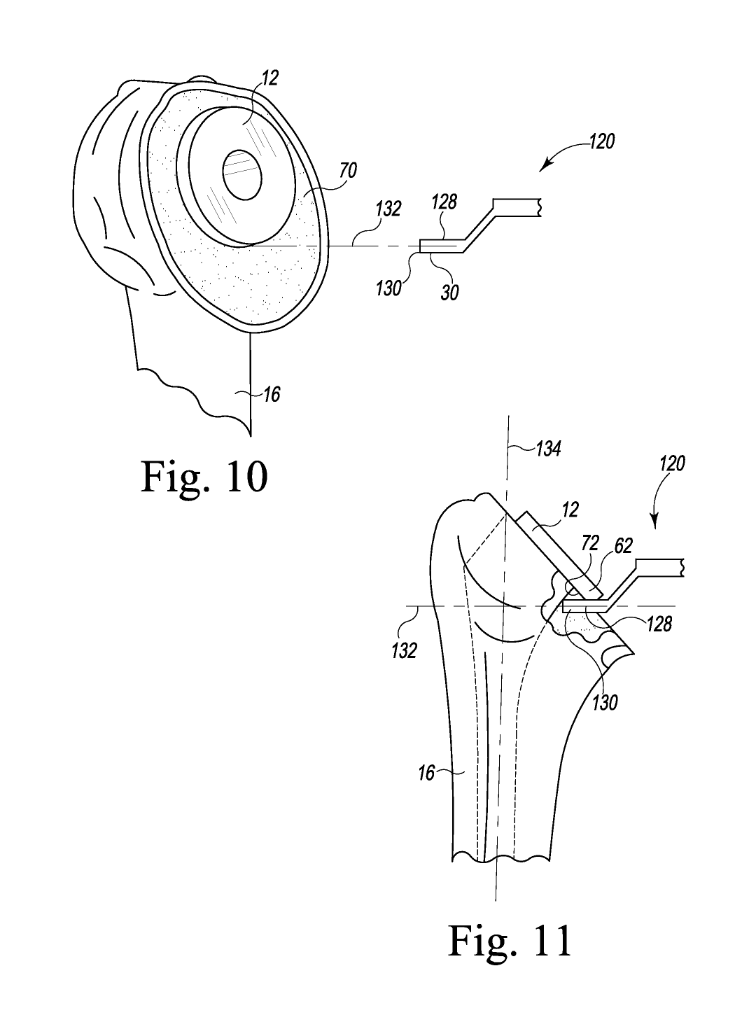

Referring now to FIGS. 10-13, another embodiment of an offset surgical tamp (hereinafter surgical tamp 120) is shown with a modified engagement tip 128 formed on its distal end 30. As shown in FIG. 10, the engagement tip 128 includes a cutting edge 130 configured to engage with the substantially planar surgically-resected medial surface 70 of the patient's humerus 16. During a revision surgery, the engagement tip 128 of the surgical tamp 120 is impacted into the medial surface 70 along an axis 132 that extends orthogonal to a longitudinal axis 134 of the humeral stem component 12. In other words, the surgical tamp 120 is impacted perpendicular to the patient's humerus 16. As shown in FIG. 11, the cutting edge 130 cuts into the medial surface 70 of the humerus 16 to position the engagement tip 128 below the collar 62 of the implanted humeral stem 12.

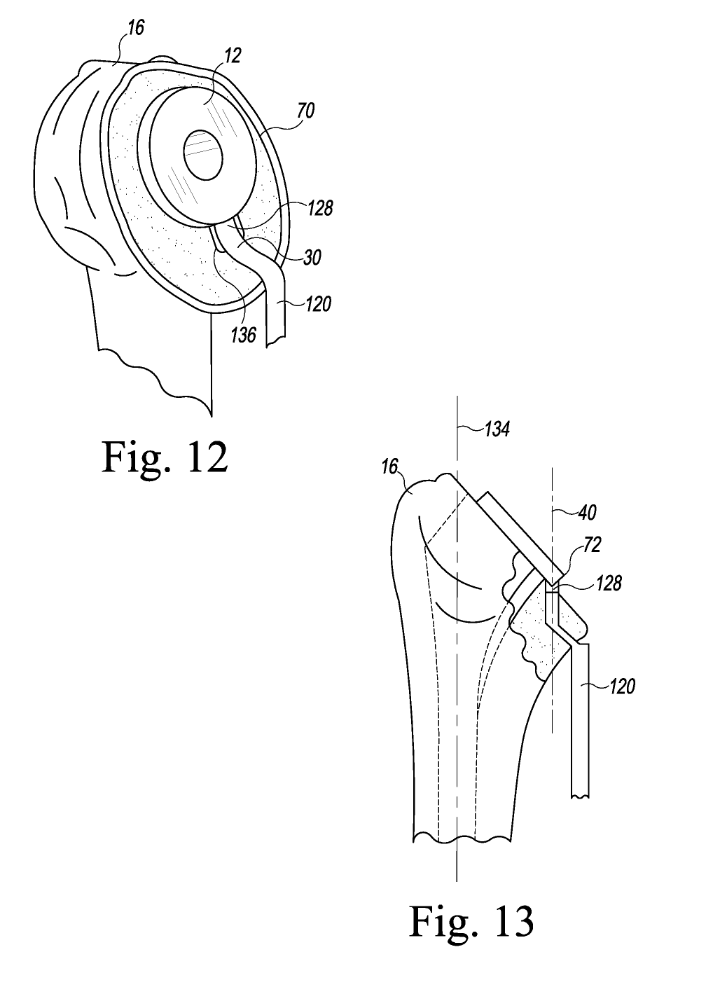

Once the engagement tip 128 is positioned below the collar 62, the surgeon may rotate the surgical tamp 120 downward to position the engagement tip 128 in contact with the inferior surface 72 of the collar 62 of the humeral stem component 12, as shown in FIGS. 12 and 13. As the surgical tamp 120 is rotated, the distal end 30 of the surgical tamp 120 is pressed into the medial surface 70, thereby forming a groove or depression 136 in the medial surface 70. When properly positioned, the longitudinal axis 40 of the surgical tamp 120 extends parallel to the longitudinal axis 134 of the humeral stem component 12 (i.e., parallel to the patient's humerus).

Once the engagement tip 128 is positioned against the inferior surface 72 of the collar 62 in such a manner, the surgeon strikes the metal strike plate 34 of the surgical tamp 120 with a surgical mallet, sledge, or other impaction tool to generate an extraction force that is applied to the inferior surface 72 of the collar 62 to urge the implanted humeral stem component 12 from the patient's humerus 16, as described above in regard to FIGS. 1-9. The surgeon may then lift away the freed humeral stem component 12 from the patient's humerus 16. The surgeon may then perform the remainder of the revision surgical procedure, including the implantation of a revision humeral stem component (not shown) into the patient's humerus 16.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A method of surgically removing an implanted humeral stem component from a humerus of a patient, comprising:
cutting a bone fragment from a medial surface of the humerus of the patient so as to expose an inferior surface of a collar of the humeral stem component,
positioning an engagement tip of a surgical tamp in contact with the inferior surface of the collar of the humeral stem component, and
impacting the surgical tamp so as to urge the humeral stem component from the humerus of the patient.

2. The method of claim 1, wherein cutting the bone fragment comprises performing an osteotomy on the medial surface of the humerus of the patient so as to cut the bone fragment from the medial surface of the humerus of the patient thereby exposing the inferior surface of the collar of the humeral stem component.

3. The method of claim 2, wherein performing the osteotomy on the medial surface of the humerus of the patient comprises advancing an osteotome into the medial surface of the humerus of the patient at a location under an inferior edge of the collar of the humeral stem component.

4. The method of claim 1, wherein:
the surgical tamp comprises a handle that is offset from the engagement tip, and
impacting the surgical tamp comprises impacting the handle of the surgical tamp so as to urge the humeral stem component from the humerus of the patient.

5. The method of claim 4, wherein impacting the surgical tamp comprises impacting the handle of the surgical tamp along a first axis so as to apply an extraction force to the humeral stem component along a second axis, the first axis being parallel to, and spaced apart from, the second axis.

6. The method of claim 1, wherein:
the engagement tip of the surgical tamp has a slot formed therein, and
positioning the engagement tip of the surgical tamp in contact with the inferior surface of the collar of the humeral stem component comprises positioning the engagement tip of the surgical tamp such that a medial fin of the humeral stem component is received into the slot.

7. A method of surgically removing an implanted humeral stem component from a humerus of a patient, comprising:
positioning an engagement tip of an offset surgical tamp in contact with an inferior surface of a collar of the humeral stem component, and
impacting the surgical tamp along a first axis so as to apply an extraction force to the humeral stem component along a second axis, the first axis being parallel to, and spaced apart from, the second axis.

8. The method of claim 7, further comprising cutting a bone fragment from a medial surface of the humerus of the patient so as to expose the inferior surface of the collar of the humeral stem component.

9. The method of claim 8, wherein cutting the bone fragment comprises performing an osteotomy on the medial surface of the humerus of the patient so as to cut the bone fragment from the medial surface of the humerus of the patient thereby exposing the inferior surface of the collar of the humeral stem component.

10. The method of claim 9, wherein performing the osteotomy on the medial surface of the humerus of the patient comprises advancing an osteotome into the medial surface of the humerus of the patient at a location under an inferior edge of the collar of the humeral stem component.

11. The method of claim 8, wherein:
the engagement tip of the surgical tamp has a slot formed therein, and
positioning the engagement tip of the surgical tamp in contact with the inferior surface of the collar of the humeral stem component comprises positioning the engagement tip of the surgical tamp such that a medial fin of the humeral stem component is received into the slot.

12. The method of claim 7, wherein:
the surgical tamp comprises a handle that is offset from the engagement tip, and
impacting the surgical tamp comprises impacting the handle of the surgical tamp so as to urge the humeral stem component from the humerus of the patient.

13. The method of claim 12, wherein impacting the surgical tamp comprises impacting the handle of the surgical tamp along the first axis so as to apply an extraction force to the humeral stem component along the second axis.

14. The method of claim 7, wherein positioning the engagement tip of the surgical tamp in contact with the inferior surface of the collar of the humeral stem component comprises impacting the surgical tamp along a third axis extending orthogonal to a longitudinal axis of the humeral stem component.

15. An orthopaedic surgical instrument for extracting an implanted humeral stem component for a patient's humerus, comprising:
a surgical tamp comprising (i) an elongated shaft having a proximal end and an opposite, distal end, (ii) a strike plate secured to the proximal end of the elongated shaft, and (iii) an engagement tip secured to a distal end of the elongated shaft, wherein: (a) the proximal end defines a first longitudinal axis, (b) the distal end defines a second longitudinal axis, and (c) the second axis is offset from and parallel to the first axis.

16. The orthopaedic surgical instrument of claim 15, wherein the elongated shaft of the surgical tamp further includes: (i) a mid-shaft section connected to and extending distally from the proximal end, (ii) a first elbow connected to the mid-shaft section, (iii) a second elbow connected to both the first elbow and the distal end of the elongated shaft.

17. The orthopaedic surgical instrument of claim 15, wherein the surgical tamp further comprises a grip positioned around the elongated shaft at a location proximate to the strike plate.

18. The orthopaedic surgical instrument of claim 15, wherein:
the engagement tip defines a third longitudinal axis, and
the third longitudinal axis is orthogonal to both the first and second longitudinal axes.

19. The orthopaedic surgical instrument of claim 15, wherein the second axis is offset from the first axis in a direction that is orthogonal to both the first and second axes.

20. The orthopaedic surgical instrument of claim 15, wherein the engagement tip of the surgical tamp has a slot formed therein.
